(19) United States
(12) Patent Application Publication
(10) Pub. No.: US 2024/0207107 A1
(43) Pub. Date: Jun. 27, 2024

(54) A BOOSTER FOR A MOISTURE MANAGEMENT PANT

(71) Applicant: MODIBODI AUSTRALIA PTY LTD, Rozelle, New South Wales (AU)

(72) Inventors: Kristy CHONG, New South Wales (AU); Alice WARNER, New South Wales (AU)

(73) Assignee: MODIBODI AUSTRALIA PTY LTD, Rozelle, New South Wales (AU)

(21) Appl. No.: 18/557,738

(22) PCT Filed: Apr. 14, 2022
(86) PCT No.: PCT/AU2022/050345

§ 371 Date: Oct. 27, 2023

(30) Foreign Application Priority Data
May 27, 2021 (AU) 2021901589
May 27, 2021 (AU) 2021901597

(51) Int. Cl. A61F 13/496; A61F 13/15; A61F 13/505; A61F 13/56; A61F 13/70

(52) U.S. Cl. CPC A61F 13/496; A61F 13/15203; A61F 13/505; A61F 13/5638; A61F 13/70; A61F 2013/15487

(57) ABSTRACT

A moisture management pant undergarment comprising booster pad which is in a friction, non-slip or interference fit arrangement with a crotch panel of an underwear garment.

### FIELD OF THE INVENTION

The invention relates to improved moisture management pant undergarments having an improved booster for additional moisture management capacity.

### BACKGROUND

Disposable moisture management pant undergarments including nappies, underpants and briefs are an environmental problem of concern. Consumers are interested in more eco-friendly products such as reusable sanitary and incontinence products, including nappies and incontinence underwear products. Cloth nappies or diapers for infants and children have become popular in recent years. However, such reusable products have drawbacks including frequent leaking, especially at night-time. While booster systems are available for some cloth nappy or underwear products, they often slip or move around during use and can even slide out of the garment, soiling the wearer, clothing and even bedding at times. Many existing booster systems are limited in terms of additional volume capacity. Often, adult incontinence products tend to be bulky and unsightly and may also struggle to manage the larger volumes of moisture associated with incontinence.

Furthermore, while cloth-based heavy duty moisture management pant undergarments such as cloth nappies and cloth incontinence underwear are known, while such cloth solutions are launderable, due to the tendency of faeces to deeply embed into the fabric fibres, they tend to stain quickly, odours are difficult to remove and they require quite harsh laundering conditions particularly involving high temperatures and/or soaking with bleach or other oxidising products. Such conditions can lead to premature failure of the garments in terms of protection from leakage.

Drying after laundering can also be a challenge, as drying can be slow due to the nature of the products which means the products can acquire a musty smell and a greater number of garments overall is required to ensure a ready supply of useable product is to hand at all times due to the slow drying process. This is particularly the case for undergarments supporting very high moisture management capabilities as they hold significant amounts of moisture when wet after laundering. Indeed, the degree of slow drying only became evident during design of the launderable, reusable nappy and incontinence undergarments described herein which have multiple layers of fluid absorbing fabric in their cores. Such a problem has not been anticipated or recognised in prior art solutions to the problem of high moisture management as is required for nappies and incontinence underwear.

In one embodiment, an improved moisture management pant undergarment comprises a pant body having a waist portion, a front panel and a back panel. The waist portion may be attached to both the front and back panels. In one embodiment, an improved moisture management pant undergarment comprises a pant body having a waist portion, a front panel and a back panel connected by a pair of side wings, and a pair of leg openings having a crotch panel disposed therebetween.

Improved moisture management pants and/or pant undergarments which address one or more of the above problems or provides a useful alternative are desirable.

### STATEMENTS OF THE INVENTION

According to a first aspect of the present invention, there is provided a launderable moisture management pant undergarment comprising:
(a) a pant body comprises leg openings having a crotch panel disposed therebetween, wherein surfaces of at least the crotch panel are lined with a first moisture wicking fabric comprising a brushed or otherwise textured surface having fibers displaced from the fabric knit, and
(b) a removeable moisture management booster pad for positioning over the lined crotch panel, the booster pad comprising:
   an external covering surrounding a fluid trapping core component of the booster pad, the external covering comprising a second moisture wicking fabric comprising a brushed or otherwise textured surface having fibers displaced from the fabric knit, and
   wherein the fluid trapping core component of the booster pad traps bodily fluid wicked away from the wearer's skin by the second moisture wicking fabric of the pad; wherein
physical contact between a first surface of the moisture wicking fabric lined crotch panel of the pant undergarment and a second surface of the moisture wicking fabric of the external covering of the booster pad, results in a friction, non-slip or interference fit between the booster pad and the moisture wicking fabric lined crotch panel.

Desirably, the pant body further comprises a waistband, a front panel and a back panel connected by a pair of side wings.

Also described herein is a moisture management pant undergarment comprising:
(a) a pant body having a waistband, a front panel and a back panel connected by a pair of side wings, and a pair of leg openings having a crotch/gusset panel disposed therebetween, wherein inner surfaces of at least the crotch/gusset panel are lined with a first moisture wicking fabric comprising a brushed or otherwise textured surface having fibers displaced from the fabric knit, and
(b) a removeable moisture management booster pad for positioning over the lined crotch panel, the booster pad comprising:
an external covering surrounding a fluid trapping core component of the booster pad, the external covering comprising a moisture wicking fabric comprising a brushed or otherwise textured surface having fibers displaced from the fabric knit, and wherein the fluid trapping core component of the pad traps bodily fluid wicked away from the wearer's skin by the moisture wicking fabric of the pad; wherein
physical contact between a surface of the lined inner of the crotch panel of the pant undergarment and a surface of the external covering of the moisture wicking fabric of the booster pad, results in a friction, non-slip or interference fit between the booster pad and the moisture wicking fabric lined crotch panel.

Desirably, the pant undergarment comprises an optional fluid trapping core component associated with the crotch panel of the pant undergarment which traps bodily fluid wicked away from the wearer's skin by the moisture wicking fabric lining of the pant which is present in at least the crotch panel of the pant undergarment. The crotch panel may also be described as a gusset panel.

A brushed or otherwise textured surface having fibers displaced from the fabric knit is one in which the fibers of the fabric have been pulled out or teased out from the knit of the fabric, typically by brushing or teasing. It has been found that brushed fabrics alleviate many of the problems described above and in particular have found to provide a non-stick surface in terms of faeces, whereby faeces more readily can be scraped or brushed off the fabric. Brushed fabrics are described elsewhere herein in more detail and example, includes a brushed polyester fabric as well as a brushed polyester composite fabric.

Suitably, one or more of the first and second moisture wicking fabrics is a brushed polyester composite or blended fabric. In one embodiment, a preferred brushed polyester composite/blend is a “polybrush”, for example, a polyester and spandex polybrush composite/blend, which more preferably contains more than 50% polyester and less than 50% spandex, and most particularly contains about 87% polyester and about 13% spandex. In another embodiment the composite/blend is 85% polybrush and 15% spandex.

According to a second aspect of the present invention, there is provided a moisture management booster pad for, preferably when positioned over, a crotch panel of a pant undergarment lined with a first moisture wicking fabric comprising a brushed or otherwise textured surface having fibers displaced from the fabric knit, the booster pad comprising:
an external covering surrounding a fluid trapping core component, the external covering comprising a second moisture wicking fabric comprising a brushed or otherwise textured surface having fibers displaced from the fabric knit,
wherein the fluid trapping core component of the booster pad traps bodily fluid wicked away from the wearer's skin by the moisture wicking fabric into the core of the booster pad; wherein physical contact between a first surface of the moisture wicking fabric lined crotch panel of the pant undergarment and a second surface of the moisture wicking fabric of the external covering of the booster pad, results in a friction, non-slip or interference fit between the booster pad and the moisture wicking fabric lined crotch panel.

The friction, non-slip or interference fit sticks the booster pad to the crotch panel of the pant undergarment to the degree that the booster pad does not slip out of place during use. This greatly reduces the risk of accidental leakage which could otherwise occur as a result of undesired pad movement/slippage out of position.

Also described herein is a moisture management booster pad insert for positioning in the crotch/gusset region of a pant undergarment lined with a first moisture wicking fabric comprising a brushed or otherwise textured surface having fibers displaced from the fabric knit, the booster pad comprising:
an external covering surrounding the core component, the covering comprising a second moisture wicking fabric comprising a brushed or otherwise textured surface comprising a brushed or otherwise textured surface having fibers displaced from the fabric knit, and
a fluid trapping core component which traps bodily fluid wicked away from the wearer's skin by the moisture wicking fabric of the pad.

According to a third aspect of the present invention, there is provided a kit comprising:
(i) one or more removeable moisture management booster pads according to the second aspect of the invention; and
(ii) one or more moisture management pant undergarments each comprising:
a pant body comprising leg openings having a crotch panel disposed therebetween, wherein inner surfaces of at least the crotch panel are lined with a further moisture wicking fabric comprising a brushed or otherwise textured surface having fibers displaced from the fabric knit.

Also described herein, the invention provides a kit comprising:
(i) one or more removeable moisture management booster pad inserts according to the second aspect; and
(ii) one or more moisture management pant undergarments each comprising:
a pant body having a waistband, a front panel and a back panel connected by a pair of side wings, and a pair of leg openings having a crotch panel disposed therebetween, wherein inner surfaces of at least the crotch panel are lined with a first moisture wicking fabric comprising a brushed or otherwise textured surface having fibers displaced from the fabric knit.

Preferably, the kit comprises two or more pads of the invention and two or more pant undergarments of the invention.

As used herein, the terms “first”, “second”, “third”, “further”, etc in relation to various features of the disclosed devices are arbitrarily assigned and are merely intended to differentiate between two or more such features that the device may incorporate in various embodiments. The terms do not of themselves indicate any particular orientation or sequence. Moreover, it is to be understood that the presence of a “first” feature does not imply that a “second” feature is present, the presence of a “second” feature does not imply that a “first” feature is present, etc.

Further aspects of the invention appear below in the detailed description of the invention.

### BRIEF DESCRIPTION OF THE DRAWINGS

Embodiments of the invention will herein be illustrated by way of example only with reference to the accompanying drawings in which:

FIG. 1 illustrates one example of a moisture management pant/undergarment of the invention in the form of a fold up nappy which in this figure is shown in the flat or unfolded configuration. FIG. 1a shows a removeable moisture management booster pad of the invention, which is positioned over the lined crotch panel in a non-slip arrangement as described herein in FIG. 1.

FIG. 2 illustrates a front on view of the fold up nappy which in this figure is shown in the folded configuration. The removeable moisture management booster pad of the invention is not visible in this figure;

FIG. 3 illustrates a rear view of the fold up nappy which in this figure is shown in the folded configuration. The removeable moisture management booster pad of the invention is not visible in this figure;

FIG. 4 illustrates the front panel of the fold up nappy when the nappy is in the unfolded configuration;

FIG. 5 illustrates (a) the shape of an exemplary floating liner assembly of the invention comprising a lining in the forms of a moisture wicking fabric having a brushed or otherwise textured surface; (b) an exploded view of the components of an example of a removeable moisture management booster pad of the invention booster pad of the invention for use with a nappy which shows the outer covering in the form of an upper covering layer and a lower covering layer which are a moisture wicking fabric having a brushed or otherwise textured surface;

FIG. 6 illustrates (a) a front on view of a pair of women's underwear having the crotch portion provided with a lining in the forms of a moisture wicking fabric having a brushed or otherwise textured surface; (b) an example of a booster pad of the invention for use with a pair of women's underwear having a surface covering which in the form of an upper covering layer and a lower covering layer which are a moisture wicking fabric having a brushed or otherwise textured surface;

FIG. 7 illustrates (a) a left side on view of a pair of women's underwear removeable moisture management booster pad of the invention being moved into position over the crotch portion of the underwear; (b) a left side on view of a pair of women's underwear having the booster pad of the invention in position in the crotch portion of the underwear wherein contact between a lined surface of the crotch panel and a surface of the moisture wicking fabric of the booster pad, results in a friction or interference fit between the booster pad and the moisture wicking fabric lined crotch panel; and

FIG. 8 illustrates (a) an exploded view of the fabric arrangement for a men's trunk style gusset layering, (b) a left side on view of the full men's trunk with polybrush lined floating liner and polybrush lining in the crotch region to stop slippage of the floating liner in use, (c) booster pad for men's undergarments with additional volume capacity to on end of the pad.

### DETAILED DESCRIPTION OF THE INVENTION

The present invention is described with reference to the following examples. It is to be understood that the examples are illustrative of, and not limiting to, the invention described herein.

As explained above while existing cloth moisture management solutions, are launderable, due to the tendency of faeces to deeply embed into the fabric fibres, particularly nappies, they tend to stain quickly, odours are difficult to remove and they require quite harsh laundering conditions particularly involving high temperatures and/or soaking with bleach or other oxidising products. Such conditions can lead to premature failure of the garments in terms of protection from leakage. The inventors have found that a brushed or otherwise textured fabric alleviates this problem as well as the problem of booster pad slippage and leaking caused by such slippage. This is particularly the case where the brushed or otherwise textured fabrics is used on the crotch portion of a pant undergarment which is likely to be more prone to soiling. Further advantages arise where a booster pad which comprises a brushed or otherwise textured fabric is used with the pant undergarment of the invention as together they work in concert to reduce slippage of a booster pad when used with the pant undergarment.

The invention relates to an improved moisture management pant undergarment for wicking bodily fluid away from a wearer's skin into a first fluid trapping core component of the pant undergarment and/or to a second fluid trapping core component of a booster pad when used with the pant undergarment. Preferred moisture management pant undergarments include a booster pad for additional moisture management capability. Suitably, the removeable moisture management booster pad is free of fastener devices for releasably attaching the booster pad to the pant body. This makes wearing the booster pad more comfortable and also avoids having to fasten and unfasten the booster pad when inserting and removing. As described elsewhere herein, the booster pad is provided with a special fabric cover of a brushed or otherwise textured fabric as defined herein alleviates the slippage problem, particularly when used with an undergarment provided with a similarly brushed or otherwise textured fabric in at least the crotch portion, that when contacted together results in a non-slip or interference arrangement between the 35 pad and the undergarment.

It will be understood that the undergarment is for wicking bodily fluid away from a wearer's skin into one or more of the first and second fluid trapping core components associated with the undergarment.

In one embodiment, the first moisture wicking fabric lining the crotch portion and the second moisture wicking fabric of the covering of the booster pad are different. However, desirably the moisture wicking fabric lining the crotch portion and the moisture wicking fabric of the covering of the booster pad are the same fabric. This advantageously is believed to maximize the interference fit and also avoids the wearer experiencing different material feels or sensations during use.

In a preferred embodiment, the invention is concerned with two interrelated/compatible components, a moisture management pant undergarment, as well as a moisture management booster pad for positioning over a crotch panel of the pant undergarment lined with a first moisture wicking fabric comprising a brushed or otherwise textured surface having fibers displaced from the fabric knit. The moisture management booster pad has an external covering that has a second moisture wicking fabric comprising a brushed or otherwise textured surface having fibers displaced from the fabric knit. To derive maximum benefit from the invention, the moisture management pant undergarment of the invention is specifically designed for use with a compatible removeable moisture management booster pad, which used in combination, result in generation of friction, non-slip or interference fit arrangement between the booster pad and a correspondingly lined crotch panel of a compatible pant undergarment. This is a significant improvement over existing booster pad-pant arrangements where unwanted movement and slippage of the booster pad occurs as a result of the wearer's normal movements. Movement and slippage of the booster pad causes the pad to slip or move out of the ideal moisture absorbing position and results in discomfort, leaks or other malfunction of the moisture management pant underwear garment. Ultimately this leads to lack of consumer confidence in the product and lost sales.

In addition to the friction or anti-slip arrangement, as a result of the components including, preferred moisture management pant undergarments of the invention and/or moisture management booster pads of the invention have a very high moisture management capability due to their unique design which utilises a number of layers of highly absorbent fluid trapping fabric provided as a fluid trapping core in the pant undergarment, the booster pad, or both. The absorbent fluid trapping fabric is described herein and is preferably a microfibre toweling fabric, e.g., a bamboo blend (e.g. a bamboo terry blend such as bamboo viscose terry, or bamboo lycocell terry) or a polyester blend (e.g. polyester/polyamide) microfibre toweling composite in the form of fluid trapping core components provided (i) in and around the crotch panel/region of the undergarment, and/or (ii) in the core of the moisture management booster pad of the invention. The moisture management pant undergarment has a very high moisture management capability due to its design which utilises a number of layers of absorbent fluid trapping fabric, preferably microfibre terry fabric, of a fluid trapping core component provided in and around the crotch panel/region of the undergarment and/or as a part of a booster pad.

Desirably, the fluid trapping core component comprises at least one layer of fabric, or at least two layers of fabric, or at least three layers of fabric, or at least four layers of fabric, or at least five layers of fabric, or at least six layers of fabric, or at least seven layers of fabric, or at least eight layers of fabric, or at least nine layers of fabric, or at least ten layers of fabric. Each layer of fabric can be the same or different or a mixed combination of fabrics. In a preferred embodiment, the fluid trapping core component comprises three layers of fabric. In other embodiments, the absorbent fluid trapping fabric is used in the form of a fabric trapping core fabric assembly of at least 1, at least 2, at least 3, at least 4 or at least 5 layers of the absorbent fluid trapping fabric. In some embodiments, the absorbent fluid trapping fabric is present in the fabric trapping core fabric assembly up to 5 layers but no more of the absorbent fluid trapping fabric. More layer causes undesirable bulkiness and reduces the wearability of the booster pad.

Desirably, the moisture management pant undergarment of the invention comprises a pant body having a waistband, a front panel and a back panel connected by a pair of side wings, and a pair of leg openings having a crotch panel disposed therebetween.

Suitably, the inner surfaces of at least the crotch panel are lined with a first moisture wicking fabric comprising a brushed or otherwise textured surface having fibers displaced from the fabric knit.

The undergarment is provided with a removeable moisture management booster pad for positioning over the lined crotch panel, the booster pad comprising (i) an external covering surrounding the fluid trapping core component, the covering comprising a (second) moisture wicking fabric comprising a brushed or otherwise textured surface having fibers displaced from the fabric knit, and (ii) a further (second) fluid trapping core component which traps bodily fluid wicked away from the wearer's skin by the moisture wicking fabric of the pad.

Suitably, the moisture wicking fabric has a weight or density in a range of about 100 gsm to about 300 gsm, more preferably between about 150 gsm and about 250 gsm, for example, 180 gsm, and more preferably still between about 215 gsm and about 245 gsm, most preferably about 235 gsm, particularly in the case of the nappy embodiment.

Preferably, the fluid trapping core component (the first and/or second fluid trapping component) comprises at least one layer of a fabric having a weight or density in a range of 50 gsm to 500 gsm, more preferably between 150 gsm and 350 gsm, and most preferably 240 gsm or 280 gsm. In other embodiments, the moisture wicking fabric has a weight or density in a range of 100 gsm to 300 gsm, more preferably between about 150 gsm and about 250 gsm, and most preferably about 180 gsm or about 235 gsm.

Each layer does not have to have the same weight or density. A combination of different weights or densities can be used if so desired.

Suitably, one or more of the first and second fluid trapping core components has a thickness of between 1 and 20 mm, preferably 1 and 15 mm, more preferably 1 to 9 mm or in underwear less than about 7 mm. The thickness can be same for both core in the pant undergarment and booster pad, or can be different.

In embodiments using a fluid trapping core component of about 500 gsm density, for example, 5 layers of a preferred fabric, such as polyester/polyamide microfibre toweling, will have a capacity of up to 500 mls of fluid. Likewise, a fluid trapping core provided having three 3 absorbent layers each of 500 gsm density (such as polyester/polyamide microfibre toweling) allows accommodation of up to 1800 mls of fluid. When used together, as a first and second core respectively (so a booster pad of the invention comprising 5 absorbent layers of 100 gsm and a pant fluid trapping core component with 3 absorbent layers of 500 gsm) allows accommodation of significant amounts of fluid, up to about 2300 mL. It will be understood that the density and number of layers as well as the particular absorbent fabric used can be adjusted to provide a variety of maximum volume capacities.

Preferred booster pads of the invention comprising 5 absorbent layers as described herein, preferably of a 280 gsm polyester/polyamide terry microfibre, may accommodate up to 250 mls of fluid. This is a useful capacity for the nappy booster in particular. However, additional capacity can be added by adding more absorbent layers as required to the booster pad. Clearly more layers add additional bulk to the booster which may be undesirable in some garments and/or to some users.

Desirably, the pant undergarment further comprises an optional further fluid trapping core component associated with the crotch panel of the pant undergarment which traps bodily fluid wicked away from the wearer's skin by the first moisture wicking fabric lining in the crotch panel region. Inclusion of a second fluid trapping core component in a booster as described herein in combination with a first fluid trapping core component in the floating liner of the pant undergarment and optionally a further fluid trapping core component in the crotch panel of the undergarment as described herein allows accommodation of significant amounts of fluid. For example, an undergarment fluid trapping core having three 3 absorbent layers, preferably of a 280 gsm terry microfibre, allows accommodation of up to 900 mL of fluid. This is a useful capacity for the nappy booster as an incontinence underpant in particular. This is also a useful capacity for the core of a nappy or incontinence underpant floating liner assembly in particular. However, it will be understood that additional capacity can be added by adding more absorbent layers as required to first and/or second fluid trapping cores. It will be noted that the greater capacity of the undergarment fluid trapping core compared to the booster is due to the greater surface area of absorbent fabric used in the undergarment crotch panel, particularly the nappy or incontinence product compared to the smaller dimensioned booster pad.

When used together, the preferred booster pads of the invention comprising 5 absorbent layers and the preferred fluid trapping core component of the pant with 3 absorbent layers as described herein allows accommodation of significant amounts of fluid. For example, when the preferred 5 layer booster pads of the invention accommodating up to 250 mL (such as comprising 5×280 gsm polyester/polyamide terry microfibre layers) are combined with an undergarment fluid trapping core having three 3 absorbent layers, preferably of a 280 gsm terry microfibre, accommodating up to 900 mL of fluid, a total absorbency of up to 1150 mL may be achieved.

Suitably, as explained above, when together, resultant physical contact results in a friction, non-slip or interference fit between the booster pad and the moisture wicking fabric lined crotch panel. The friction or otherwise non-slip arrangement thus formed helps prevent the removeable moisture management booster pad of the invention from slipping and moving around within the undergarment when worn by the wearer. The effect of the friction, non-slip or interference fit is clearly evident through the obvious increase in force required to move each component against each other. The effect is also evident from the observation of significantly less slippage of the booster pad out of position or even out of the pant when being worn and subjected to movement of the wearer.

Preferably, the inner (skin/body facing) surfaces of at least the crotch panel of the undergarment are lined with a brushed or otherwise textured or roughed surface. In use, the removeable moisture management booster pad is positioned over the lined brushed wicking fabric of the crotch panel, the booster pad comprising a fluid trapping core component and an external brushed covering surrounding the core component. A friction fit, an interference fit or an otherwise anti-slip arrangement is formed between the wicking fabric covering of booster pad and the moisture wicking fabric lined crotch panel. The fit can be felt by hand as a notable hand force needs to be applied to move one component over the other when they are in surface contact. The invention is useful as reduced and controlled booster pad movement aids in reducing the risk of malfunction of the booster for example resulting in unwanted leaking as a result of slipping/moving out of the desired position. Further, such fastener devices are fiddly and tricky to use, especially in the case of carers/parents needing to attach, or those with disabilities

Desirably, the removeable moisture management booster pad is otherwise free of fastener devices for releasably attaching the booster pad to the pant body. Fastener devices include hook & loop tape, press studs, buttons and the like. Such devices are not needed due to the nature of the friction fit, the interference fit or the otherwise anti-slip arrangement which is formed on surface contact between the wicking fabric covering of booster pad and the moisture wicking fabric lined crotch panel. Fastener devices are advantageously thus not required in the garment/components of invention, which is desirable as they may feel uncomfortable to the wear and can lose function or fail after time.

A fabric having a brushed or otherwise textured or roughened surface will typically be one having brushed or otherwise raised surface fibers, particularly having fibers displaced from the plane of the fabric knit or weave. For example, the fibers of the fabric will have been pulled out or teased out from the knit of the fabric, typically by brushing or combing. The brushed fibres generally create a soft and napped texture on the fabric surface. For example, the fabric may have undergone a mechanical brushing process in which fine, metal brushes carefully rub the fabric to produce fine fibres from the woven yarns, creating extra softness on the surface of the fabric. The brushing produces fibers which are teased out from the surface of the fabric to provide the brushed, peached, textured or otherwise roughened surface of the wicking fabric as required by the present invention.

Desirably, a preferred undergarment is capable of wicking bodily fluid away from a wearer's skin into one or more of the first and second fluid trapping core components associated with the undergarment and booster. Thus, while the second fluid trapping core component associated with the crotch panel is optional, in a preferred embodiment, the second fluid trapping core component is present in the crotch portion of the pant undergarment, as it provides an inherent moisture management capacity to the garment beyond that provided by the booster pad. Coupled with the booster pad of the invention, the combination provides particularly high moisture management capability. Depending on the size of booster pad used, it enables tailorable moisture management capability, which may be desirable in certain circumstances.

Also described herein is a standalone moisture management booster pad for positioning over a crotch panel of a pant undergarment lined with a first moisture wicking fabric comprising a brushed or otherwise textured surface having fibers displaced from the fabric knit, the booster pad comprising:
an external covering surrounding the core component, the covering comprising a second moisture wicking fabric comprising a brushed or otherwise textured surface comprising a brushed or otherwise textured surface having fibers displaced from the fabric knit, and
a fluid trapping core component which traps bodily fluid wicked away from the wearer's skin by the moisture wicking fabric of the pad. The pad may be sold alone, together with a pant undergarment or as part of a kit as described herein.

As the component parts are intended to be used together to obtain the described advantages of the invention, the invention further extends to a kit comprising the components of the invention. In one embodiment, the kit comprises: (i) one or more removeable moisture management booster pads according to the second aspect; and (ii) one or more moisture management pant undergarments each comprising: a pant body having a waistband, a front panel and a back panel connected by a pair of side wings, and a pair of leg openings having a crotch panel disposed therebetween, wherein inner surfaces of at least the crotch panel are lined with a first moisture wicking fabric comprising a brushed or otherwise textured surface having fibers displaced from the fabric knit. In one embodiment, the kit comprises: (i) one or more removeable moisture management booster pads as described herein; and (ii) one or more moisture management pant undergarments each comprising: a pant body comprising a pair of leg openings having a crotch panel disposed therebetween, wherein inner surfaces of at least the crotch panel are lined with a first moisture wicking fabric comprising a brushed or otherwise textured surface having fibers displaced from the fabric knit.

For example, a preferred kit comprises (i) one or more removeable moisture management booster pad for positioning over a lined crotch panel of a pant undergarment, the booster pad comprising an external covering surrounding the core component, the covering comprising a second moisture wicking fabric comprising a brushed or otherwise textured surface, and a first fluid trapping core component which traps bodily fluid wicked away from the wearer's skin by the moisture wicking fabric of the pad; and (ii) one or more moisture management pant undergarment comprising: a pant body comprising a pair of leg openings having a crotch panel disposed therebetween, such as a pant body having a waistband, a front panel and a back panel connected by a pair of side wings, and a pair of leg openings having a crotch panel disposed therebetween, wherein inner surfaces of at least the crotch panel are lined with a first moisture wicking fabric comprising a brushed or otherwise textured surface, and an optional second fluid trapping core component associated with the crotch panel of the pant undergarment which traps bodily fluid wicked away from the wearer's skin by the first moisture wicking fabric lining at least the crotch panel, wherein in use physical contact between a lined surface of the crotch panel of the pant undergarment and a covering surface of the moisture wicking fabric of the booster pad, results in a friction, non-slip or interference fit between the booster pad and the moisture wicking fabric lined crotch panel.

To derive optimum benefit afforded by the invention, suitably, the moisture wicking fabric lining the crotch portion and the moisture wicking fabric of the covering of the booster pad can be the same. However, in some embodiments, the moisture wicking fabric lining the crotch portion and the moisture wicking fabric of the covering of the booster pad are different as long as the fabrics comprise a brushed or otherwise textured surface having fibers displaced from the fabric knit which is sufficient to form the friction or anti-slip fit of the invention.

Preferably, the moisture wicking fabric (of either or both the pant and/or the pad) comprises a brushed wool, particularly a brushed Merino wool, a brushed wicking polyester, a brushed wicking polypropylene, a brushed wicking nylon, a brushed wicking micromodal, a brushed wicking bamboo, a brushed cellulosic fabric, a brushed cotton, or composites or blends thereof. Biodegradable or recycled versions of these materials are particularly preferred. In one embodiment, the first and/or second moisture wicking fabric is a brushed fabric comprising a brushed polyester or a brushed polyester composite, preferably containing more than 50% polyester and less than 50% spandex, most preferably about 85% polyester and about 15% spandex. In one embodiment a brushed polyester composite of polyester and spandex may be used, for example, about 87% polyester and about 13% spandex. In other embodiments, non-brushed fabrics such as cotton or cellulosic fabrics may be used, provided they have an otherwise textured surface having fibers displaced from the fabric knit which is sufficient to form the friction or anti-slip fit of the invention.

In some embodiments of the pant undergarment of the invention, at least a portion of the crotch panel is overlaid with a floating liner/gusset fixedly attached to the pant body and adapted to accommodate the optional second fluid trapping core component when present and which traps bodily fluid wicked away from the wearer's skin, wherein at least one or more portions of the floating liner and the crotch/gusset panel can be physically separated from each other to expose the optional second fluid trapping core component to aid drying. Desirably, the floating liner/gusset is partially fixedly attached to the pant body enabling physical separation of the floating liner/gusset and the crotch/gusset panel.

In some embodiments, the floating liner assembly is permanently attached at one end to a front panel of the pant body and permanently attached at an opposing end to a back panel of the pant body.

Desirably, the floating liner assembly has opposing short edges and opposing long edges, preferably wherein the opposing long edges of the floating liner assembly are unattached to the pant body. Suitably, long edges of the floating liner assembly proximate the leg openings of the pant body are elasticated. In some embodiments, the fluid wicking core is permanently attached at to the floating liner assembly.

Desirably, the opposing long edges are located around the area of the leg opening in the crotch region of the garment. Desirably, the floating liner assembly is attached, preferably permanently attached at one end (e.g., the front panel) of the pant body and attached at the opposing end (e.g., to the back panel) of the pant body. Suitably, the short edges of the floating liner are attached at one end to the front panel of the pant body and attached at the opposing end to the back panel of the pant body. Preferably, the short edges of the floating liner are attached at one end to a front portion of a gusset of the pant body and attached at the opposing end to a back portion/area of the gusset of the pant body. In one embodiment, one short edge of the floating liner assembly is attached to the pant underwear, corresponding to an edge adjacent the front panel or back panel of the pant underwear. In another embodiment, two short edges of the floating liner assembly are attached to the pant underwear, corresponding to one short edge adjacent the front panel and one short edge adjacent the back panel of the pant underwear. Desirably, long edges of the floating liner are unattached to the pant body around the leg opening region. This enables good separation between the floating liner assembly and crotch panel of the pant undergarment during laundering and particularly drying when wet after laundering. This also advantageously ensures good stability of the floating liner assembly in the crotch region of a wearer during use.

Suitably, the fluid trapping core component may be attached to an underside of the floating liner assembly, the underside being distal to a wearer's skin in use. Suitably, at least a portion of the floating liner assembly comprises a fluid wicking fabric for contacting a wearer's skin in use. Desirably, the portion of the floating liner assembly comprising the fluid wicking fabric is a top side of the floating liner assembly, the topside being adjacent a wearer's skin in use.

Also described is a launderable moisture management pant undergarment for wicking bodily fluid away from a wearer's skin into a fluid trapping core component of the pant undergarment, the undergarment comprising:
a pant body comprising leg openings having a crotch panel disposed therebetween,
wherein at least a portion of the crotch panel is overlaid with a floating liner assembly partially fixedly attached to the pant body, wherein the fluid trapping core component is permanently attached to the floating liner assembly which traps bodily fluid wicked away from the wearer's skin,
wherein at least a portion of the floating liner assembly is detached from to the pant body to allow physical separation of the floating liner assembly from the crotch panel to aid drying of the fluid trapping core component.

Suitably, the fixture of the floating liner assembly to the pant body may be a permanent fixture such that the floating liner assembly cannot be removed or extracted from the pant body by the user in normal operations/use of the undergarment. Advantageously, this means that the floating liner is a permanent feature of the undergarment such that it cannot be lost or exchanged for a different floating liner from another undergarment. Preferably the fluid trapping core component is permanently attached to the floating liner, preferably to the underside of the floating liner. However, in some embodiments, the fluid trapping core component may be removably attached to the floating liner.

Desirably, the fluid trapping core component is attached to an underside of the floating liner assembly. For example, the fluid trapping core component may be attached by stitching or adhesive to the floating liner assembly. In other embodiments, the the fluid trapping core component may be attached to the floating liner by insertion into a pocket provided in the floating liner. Thus preferably the fluid trapping core component is in a pocket provided in the floating liner assembly. Conveniently, this may allow the fluid trapping core component to be replaced without removal of the garment, for example at night-time when the wearer is asleep or at other times when removal of the garment is not possible. However, it is preferred that the fluid trapping core component be permanently attached to the floating liner assembly. In embodiments where the fluid trapping core component is inserted in a pocket provided in the floating liner assembly, permanent attachment of the fluid trapping core component may be by means stitching, adhesive, or other means of attachment of the fluid trapping core component inside the pocket, either to one or more faces or one or more edges of the pocket, or may be by sealing the pocket at its edges by stitching, adhesive, or other means with the fluid trapping core component already inside it, such that the fluid trapping core component is unable to be removed from the pocket.

It will be understood that the portion of the floating liner assembly that is detached from the pant body is, during drying, intended to be arranged so as to be free of contact with the crotch panel of the pant body. This means the floating liner assembly and crotch panel are physically positioned away from each other to aid drying through enhanced drying air contact particularly with the fluid trapping core component. In use, this involves the user, after laundering the undergarment, gently pulling or teasing the wet floating liner assembly and the wet crotch panel away from each other while air drying. As used herein, the term “detached from” will be understood to be synonymous with “unattached to” in relation to the floating liner assembly and the pant body. The term “floating liner assembly” has the same meaning herein as “floating gusset liner assembly”. As used herein, the term “floating liner assembly” shall be understood being synonymous with the term “floating liner/gusset”. As used herein, the term “crotch panel” shall be understood as being synonymous with the term “crotch/gusset panel”. Desirably, the floating liner assembly is partially fixedly attached to the pant body enabling physical separation of the floating liner assembly and the crotch panel.

Desirably, at least one or more portions of the floating liner assembly are unattached to the pant body allowing physical separation of the fluid trapping core component from the crotch panel. Suitably, the floating liner is attached at one end to the front panel of the pant body and attached at the opposing end to the back panel of the pant body. To clarify, it is the portion of the liner/gusset around the leg region that is unattached to the pant body.

Advantageously, physical separation of these components allows drying air to better access the fluid trapping core component which can hold significant amounts of moisture and can be a challenge to air dry rapidly for that reason, particularly when moisture absorbent core fabrics are used. By enabling physical separation of the majority of the floating liner assembly, the improved access to drying air dramatically improves the air-drying times of these products. Faster drying products are more favoured by the consumer and also are associated with reduced risk of mould growth and malodors. At the same time, given the permanently fixed attachment to the pant body, it is impossible to lose or misplace the floating liner assembly component and the fluid trapping core component associated with the floating liner assembly.

Desirably, at least a portion of the crotch panel includes a moisture barrier component to prevent moisture leaking outside the pant during use.

Also described herein is a launderable moisture management pant undergarment for wicking bodily fluid away from a wearer's skin into a fluid trapping core component of the pant undergarment, the undergarment comprising:
a pant body comprising leg openings having a crotch panel disposed therebetween,
wherein at least a portion of the crotch panel is overlaid with a floating liner assembly partially fixedly attached to the pant body, wherein the fluid trapping core component is permanently attached to the floating liner assembly which traps bodily fluid wicked away from the wearer's skin,
wherein at least a portion of the floating liner assembly is detached from the pant body to allow physical separation of the floating liner assembly from the crotch panel to aid drying of the fluid trapping core component; and
further comprising a removeable moisture management booster pad, wherein the booster pad comprises a fluid trapping booster core component sandwiched between layers, preferably two layers, of a moisture wicking fabric booster component. Preferably the floating liner and/or the crotch part of the pant are provided with the brushed fabric of the invention.

Desirably, the moisture wicking fabric of the booster pad is the same as the moisture wicking fabric of the pant undergarment of the first aspect. Suitably, the moisture wicking fabric booster component comprises a brushed fabric having a brushed or otherwise textured surface having fibers displaced from the fabric knit, preferably a brushed polyester fabric. The removeable moisture management booster pad is preferably launderable.

Also described herein is a kit comprising: (i) one or more a launderable moisture management pant undergarments for wicking bodily fluid away from a wearer's skin into a fluid trapping core component of the pant undergarment, the undergarment comprising: a pant body comprising leg openings having a crotch panel disposed therebetween, wherein at least a portion of the crotch panel is overlaid with a floating gusset liner assembly partially fixedly attached to the pant body, wherein the fluid trapping core component is permanently attached to the floating liner assembly which traps bodily fluid wicked away from the wearer's skin, wherein at least a portion of the floating liner assembly is detached from to the pant body to allow physical separation of the floating liner assembly from the crotch panel to aid drying of the fluid trapping core component; and (ii) one or more removeable moisture management booster pads, each booster pad comprising a fluid trapping booster core component sandwiched between layers, preferably two layers, of a moisture wicking fabric booster component.

In some embodiments, a second fluid trapping core component is attached to an upper side of the crotch panel, the upper side of the crotch panel being proximal to the wearer's skin in use. Desirably, wherein the second fluid trapping core component is permanently attached to the upper side of the crotch panel or the second fluid trapping core component is removably attached to the crotch panel. If desired, the second fluid trapping core component is in a pocket provided in the crotch panel.

Suitably, the fluid trapping core component and/or the second fluid trapping core component comprises one or more layers of one or more layers of an absorbent, quick drying fabric, preferably an absorbent, quick drying microfibre fabric such as a polyester microfibre fabric or an absorbent, quick drying fabric manufactured from a cellulosic material, preferably hemp, bamboo. Desirably, the fluid trapping core component and/or the second fluid trapping core component comprises a microfibre toweling comprising greater than 50% polyester and less than about 50% polyamide or a terry toweling such as bamboo terry or bamboo terry blends including bamboo viscose terry or bamboo lyocell terry, as this material will be biodegradable/compostable in active soil. Suitably, the fluid trapping core component and/or the second fluid trapping core component comprise a microfibre toweling comprising about 80% polyester and about 20% polyamide. In some embodiments, the fluid trapping core component and/or the second fluid trapping core component comprises an absorbent, quick drying fabric manufactured from a cellulosic material, preferably hemp, bamboo, or cotton or a blend comprising hemp, bamboo, and/or cotton. In some embodiments, the fluid trapping core component and/or the second fluid trapping core component described herein comprise a synthetic fabric (derived from manmade fibres), a semi-synthetic fabric (derived from at least some fibres/raw materials that are of natural origin, e.g., cellulosic fibres) or a natural fabric (derived from fibres of 100% natural origin). Semi-synthetic fabrics refer to those fabrics being synthetically modified derivatives of a natural fabric and include fibres such as Modal®.

In a preferred embodiment, trapping core component fabrics are recycled/biodegradable/compostable such as a bamboo terry, a polyester, a recycled polyester or biodegradable polyester or a biobased polyester, or a polyester composite microfibre fabric, such as a polyester/polyamide blend. In one embodiment, the fluid trapping core component comprises a microfibre fabric comprising a polyester/polyamide blend, such as 90% polyester/10% polyamide, or 85% polyester/15% polyamide, or 80% polyester/20% polyamide, or 70% polyester/30% polyamide. In some embodiments, the polyester is a recycled polyester. In some embodiments, the polyamide is a recycled polyamide. In some embodiments, the fluid trapping core component comprises a microfibre fabric comprising recycled polyester/recycled polyamide blend, such as 90% recycled polyester/10% recycled polyamide, or 85% recycled polyester/15% recycled polyamide, or 80% recycled polyester/20% recycled polyamide, or 70% recycled polyester/30% recycled polyamide. As used herein, the term “biodegradable” is used in the sense of being at least 80% compostable but more preferably at least 95% to 100% compostable in active soil in 5 years or less, preferably in 4 years or less, preferably in 3 years or less, more preferably in 2 years or less, and most preferably in 1 year or less. Active soil comprises typical soil microbes including bacteria and fungi. As used herein, the term “compostable” refers to materials that readily break down quickly and safely in a composting system through the action of microbial activity in a defined time period to form compost or humus comprising natural materials only which cause no harm to the environment. As used herein, the term “biobased” refers to the source of raw materials for the fabric being plant-based rather than fossil-fuel based. Desirably, the fluid trapping core component comprises an absorbent, quick drying fabric, for example, manufactured from a cellulosic material, preferably hemp, bamboo, or cotton which are more environmentally friendly. Accordingly, in some embodiments, the fluid trapping core component may comprise a partly or fully bio-based absorbent quick drying fabric, a biodegradable absorbent quick drying fabric, or a compostable absorbent, quick drying fabric.

In some embodiments, at least a portion of the crotch panel includes a moisture barrier component to prevent moisture leaking outside the pant during use.

Desirably, the pant body further comprises a fluid trapping core component which traps bodily fluid wicked away from the wearer's skin.

Preferably, at least one or more portions of the floating liner are unattached to the pant body allowing physical separation of a fluid trapping core component from the crotch portion. In some embodiments, edges of pant body at the leg openings are elasticated. Suitably, the long edges of the floating liner are elasticated for comfort and additional protection though in men's and women's underwear the elastic may be omitted if desired. Suitably, the optional second fluid trapping core component is attached to the floating liner or to an upper side of the crotch portion. The fluid trapping core component may be attached by stitching or adhesive. Preferably, the fluid trapping core component is attached to the underside of the floating liner or an optional second fluid trapping core component removably attached to the floating liner. The core component may be attached to the floating liner by stitching or adhesive. Desirably, the fluid trapping core component is removably attached to the floating liner. Suitably, at least a portion of the floating liner comprises the first fluid wicking fabric as described herein which comprises a brushed or otherwise textured surface.

In some embodiments, the external covering surrounding the core component of the booster pad comprises a brushed fabric or otherwise textured/roughened fabric surface having fibers displaced from the fabric knit. In some embodiments, one or more of the floating liner assembly and the crotch panel of the pant may also comprise the brushed fabric or otherwise textured fabric surface having fibers displaced from the fabric knit. In certain preferred embodiments, both the floating liner assembly and the crotch panel comprise the brushed fabric or otherwise textured fabric surface having fibers displaced from the fabric knit, as this will prevent the floating liner from slipping around during use.

It has been found that brushed fabrics alleviate many problems such as slippage of boosters inside undergarments and consequential moisture leakage and have also been found to provide a non-stick surface in terms of faeces, whereby faeces more readily can be scraped or brushed off the fabric

In a preferred embodiment, the fluid trapping core component comprises one or more layers of an absorbent, quick drying fabric, preferably an absorbent, quick drying microfibre fabric such as a polyester, recycled or biodegradable polyester, or polyester composite microfibre fabric. Desirably, the fluid trapping core component comprises an absorbent, quick drying fabric, for example, manufactured from a cellulosic material, preferably hemp, bamboo, or cotton. Desirably, the fluid trapping core component comprises at least one layer of fabric, or at least two layers of fabric, or at least three layers of fabric, or at least four layers of fabric, or at least five layers of fabric, or at least six layers of fabric, or at least seven layers of fabric, or at least eight layers of fabric, or at least nine layers of fabric, or at least ten layers of fabric. Each layer of fabric can be the same or different or a mixed combination of fabrics. In a preferred embodiment, the fluid trapping core component comprises three layers of fabric.

Preferably, the fluid trapping core component of the crotch portion, when present, has a thickness of between 1 and 10 mm, preferably 1 to 4 mm. Preferably, the fluid trapping core component of the booster pad has a thickness of between 1 and 10 mm, preferably 1 to 4 mm. Suitably, the one or more of the first and second fluid trapping core components has a thickness of between 1 and 20 mm, preferably 1 and 15 mm, more preferably 1 to 9 mm or in underwear less than about 7 mm. In one preferred embodiment for a nappy for example, a preferred booster pad has a thickness of about less than about 2 cm, preferably about 1.7 cm.

The booster pads of the invention may have a variety of different shapes. For example, the nappy booster pad may have a rectangle shape that covers both male and female baby leaks. A booster pad for a women's undergarment or pant has curved edges similar to the shape of sanitary pad shape and also to fit neatly around legs in the underwear. A booster pad for a men's undergarment or pant has an unsymmetrical front and back shape as more protection from leaks is needed in the front portion of the undergarment. In all cases, a general increase in overall dimensions of the various booster pads will provide a ‘maxi’ version of the booster where the length or width is increased, in any embodiment.

Desirably, at least a portion of the crotch panel includes a moisture barrier component to prevent moisture leaking outside the pant during use. Preferably, the undergarment has a front panel and a back panel. In some embodiments, the undergarment also has side wings. Suitably, one or more of the front panel, a back panel, and crotch portion, and side wings, if present, comprise a moisture barrier component. Most preferably, all of the front panel, a back panel, and crotch/gusset panel, and side wings, if present, comprise a moisture barrier component. In some embodiments, the entire surface of the undergarment comprises a moisture barrier component. Suitably in a nappy or certain incontinence products, all of the front panel, a back panel, and crotch/gusset panel, and side wings, if present, comprise the moisture barrier component. In contrast, in adult and kids “regular” (non-incontinence) underwear, the moisture barrier component may be confined to the crotch/gusset panel, for example, for aesthetic and/or comfort reasons. Preferably, during use, the moisture barrier component is distal to a wearer's skin.

Preferably, the moisture barrier component comprises a moisture proof fabric, a moisture resistant fabric or a moisture repellent fabric or a fabric treated to be moisture proof, moisture resistant or moisture repellent. Preferably, the moisture barrier component is a fabric which comprises a waterproof or water-resistant laminate film or a chemical coating, rendering the fabric moisture proof fabric, a moisture resistant fabric or a moisture repellent. Anti-microbial and/or anti-odour treatments such as are known to the skilled person can used on one or more of the fabric components described herein. Preferably, the moisture barrier component comprises a waterproof, water resistant or water repellent fabric or fabric treated to have such properties which prevents movement of bodily fluids from the fluid trapping core to outside the pant, thereby avoiding leakage of captured bodily fluid. Preferably, the moisture barrier component comprises a fabric which is itself waterproof, water resistant or water repellent. Desirably, the fabric may be treated with one or more agents for example, comprising hydrophobic polymers, such as polyethylene based hydrophobic polymers, hydrophobic non-fluorinated acrylic polymers, hydrophobic non-fluorinated urethanes, or bio-based or bio-derived hydrophobic polymers or compounds, (ideally not comprising fluorinated compounds such as PFOAs), which may assist with waterproofing, water resistance or water repellence. Suitable moisture barrier films can comprise water proof, water repellent agents, compounds or compositions such as those available Huntsman or Rudolf. A preferred agent used to treat fabric to impart waterproof or water resistance or water repellency include Aquapel™ by Nanotex which is a water repellent hydrocarbon based composition. Suitable moisture barrier films having inherent waterproofing or water resistance or water repellency are available from Ding Zing. It will be understood that the terms “moisture” and “water” is used herein in the sense that it encompasses bodily fluids, particularly, sweat, urine, discharge, and/or blood.

Desirably, moisture barrier component comprises a polyester/polyurethane laminate fabric, preferably in the form of a cotton or a polyester knit fabric bonded to a polyurethane breathable and waterproofing film, generally having a thickness of 1 mm or less. In some embodiments, the moisture barrier component may comprise two layers of a polyester sandwiching a polyurethane laminate layer. Preferred moisture barrier components are recycled and/or biodegradable, for example, recycled and/or biodegradable polyester and biodegradable polyurethane which may be laminated directly onto the fluid trapping core component, the crotch panel, the front panel, the back panel, and/or the side wings. In other embodiments, the moisture barrier component comprises a biodegradable and/or compostable polymer film alone, such as a thermoplastic polyurethane (TPU) film, e.g., available from Ding Zing. Such films can be used wherever water barrier function is required, for example, it can be laminated directly to the underside of the fluid trapping core component fabric.

Preferably, when present, at least a portion of the floating liner/gusset comprises a fluid wicking fabric. Preferably, the fluid wicking fabric is provided on skin facing side of the floating liner/gusset. Direct contact with the skin allows any moisture to be quickly wicked away. The fluid wicking fabric functions to draw or wick moisture, e.g., sweat, blood, urine, quickly away from the wears skin and into the fluid trapping core component associated with the liner or the crotch/gusset panel of the pant underwear of the invention. Preferably, the inner surfaces of one or more of the front panel, the back panel, the side wings and the crotch/gusset panel are lined with a moisture wicking fabric. Preferably, the inner surfaces of each of the undergarment, such as front panel, back panel, side wings (if present) and the crotch/gusset panel are lined with a moisture wicking fabric. For some component parts, e.g., the front panel, the back panel, the side wings and the crotch/gusset panel, the moisture wicking fabric overlies the moisture barrier component. Suitably in a nappy or certain incontinence products, all of the front panel, a back panel, side wings (if present) and crotch/gusset panel comprise the moisture wicking fabric. Desirably, at least a portion of the crotch/gusset panel includes the fluid wicking fabric. However, in adult and kids “regular” (non-incontinence) underwear, the moisture wicking fabric may be confined to the crotch/gusset panel, for example, for aesthetic and/or comfort reasons.

Preferably, the moisture wicking fabric is a brushed fabric. Desirably, the moisture wicking fabric is a brushed polyester or a brushed polyester composite fabric. A polybrush fabric is particularly preferred as it resists faeces embedding in the fibres and assist in use of an anti-slip booster pad. Polybrush and other similar desirable fabrics have a surface without excessive undulations and/or piling (particularly after laundering) and overall has a tighter knit and overall a smoother, more level pile appearance than for example fleece which is often used as a nappy liner material and into which faeces readily embeds. The properties of the polybrush, in particular, allow faeces to be readily removed from the fabric during washing which means less vigorous washing/temperature cycles are sufficient prolonging the lifetime of the undergarment. In some embodiments, the polybrush comprises a blend of polyester and elastane, such as 80% polyester/20% elastane, 85% polyester/15% elastane, or 90% polyester/10% elastane. In some embodiments, the polyester in the polybrush is recycled polyester, such as an 85% recycled polyester/15% elastane blend. In some embodiments, the elastane is biobased elastane, being wholly or in part derived from a natural source such as a plant source, e.g., corn. As noted above, in some embodiments, non-brushed fabrics such as cotton or cellulosic fabrics may be used, provided they have an otherwise textured surface having fibers displaced from the fabric knit which is sufficient to form the friction or anti-slip fit of the invention, such as by physical treatment or modification of the fabric.

Preferably, the external portions of the undergarment, such as one or more of the front panel, the back panel, the side wings (if present) and the crotch panel, comprise one or more shell fabrics. Preferably, each of the front panel, the back panel, the side wings (if present) and the crotch panel comprise a shell fabric. Suitable shell fabrics include polyester, nylon (or biodegradable/recycled versions thereof), cotton, hemp, Tencel, bamboo viscose or bamboo Lyocell, or blends of polyester, nylon (or biodegradable/compostable/recycled versions thereof), cotton, hemp, Tencel, bamboo viscose or bamboo Lyocell. It will be understood that such fabrics may include blends with elastane. In some embodiments, the elastane is biobased elastane, being wholly or in part derived from a natural source such as a plant source, e.g., corn. The shell fabric may be provided with any desired pattern or colour scheme that may be desired.

In preferred embodiments, the moisture barrier component is provided between the shell fabric and the moisture wicking fabric. For example, in one embodiment, the moisture barrier component may be provided as a laminated sheet disposed between the shell fabric and the moisture wicking fabric, such as in the form of a polyurethane laminate film. In other embodiments, the moisture barrier component may be provided as a laminated sheet sandwiched between two layers of the shell fabric. Desirably, the moisture barrier component comprises a polyester/polyurethane laminate fabric, preferably in the form of a polyester knit fabric bonded to a polyurethane breathable and waterproofing film, generally having a thickness of 1 mm or less. In some embodiments, the moisture barrier component may comprise two layers of a polyester sandwiching a polyurethane laminate layer. In other embodiments, the moisture barrier component may be in the form of a polyurethane film, preferably a biodegradable/compostable thermoplastic polyurethane such as those available under the Ding Zing supplier/brand name. Such films avoid the need for polymer laminated fabrics which may be less breathable and/or environmentally friendly.

One or more components of the pant undergarment of the invention may be air permeable and/or breathable. A preferred pant undergarment of the invention is hand and/or machine washable. A preferred pant undergarment of the invention has antimicrobial and/or odour minimising properties. One or more components of the pant undergarment may comprise one or more odour elimination treatments such as one or more of Sciessent Lava XL and Agion. Furthermore, a preferred pant undergarment of the invention solves the problem of odours arising from bodily fluids as well as keeping the wearer's skin dry.

Preferably, the fluid wicking component comprises a fabric which is derived from a natural fibre or a synthetic fibre. Natural fibres are preferred. Desirably, the fabric of the fluid wicking component may be hydrophilic or may comprise a hydrophilic material. Such materials may aid in wicking away bodily fluid away from the wearer's skin. Desirably, the fabric of the fluid wicking component may comprise keratinous fibres or other moisture-wicking or which are pre-treated to enhance the moisture-absorbing capabilities thereof. Preferred keratinous fibres are stain resistant. Otherwise, is desired, the fibres may be treated to render them stain resistant, with for example, Scotchguard™ or Teflon™.

Preferably, the fabric of the fluid wicking component is in the form of a knitted or a woven fabric, preferably knitted. Preferably, the brushed fluid wicking component includes an antimicrobial material or itself comprises fibres having antimicrobial properties, for example, such as wool, preferably a soft wool such as Merino wool. It will be understood that an antimicrobial material is one that has antibacterial, antiviral, and/or antifungal properties. The antimicrobial property minimises, and more preferably eliminates, odour arising from the bodily fluid. Suitably antimicrobial materials include silver ions.

Suitably, the pant undergarment is in the form of a nappy, an incontinence brief, incontinence underwear, incontinence pantyhose, incontinence leggings, incontinence gym shorts, or toilet-training garments. The pant undergarment may also be a men's or women's briefs or underpants or trunks or shorts. The pant undergarment may also be in the form of a girls and boys teen/tween incontinence underwear, or adaptive styles for those with disabilities. Suitably, the pant undergarment is a launderable nappy, a launderable incontinence brief, launderable incontinence underwear, a launderable incontinence trunk, launderable incontinence pantyhose, launderable incontinence leggings, launderable incontinence sports shorts, such as gym, cycle or running shorts, launderable toilet-training garments, launderable nightwear garments, launderable adaptive underwear or launderable tween/teen incontinence underwear or the like. The pant undergarment may also be in the form of a launderable period management garment, such as a launderable period management brief, launderable period underwear, a launderable adaptive period management brief, launderable period management shorts, launderable period management pantyhose, or launderable period management leggings. In one embodiment, the pant undergarment as described herein is not a launderable period management garment. In one embodiment, the pant undergarment as described herein is a launderable nappy or a launderable incontinence garment. In one embodiment, the pant undergarment as described herein is a launderable nappy, a launderable incontinence garment, or a launderable period management garment.

Desirably, the pant undergarment is in a pull up confirmation or in a foldable configured to be folded into a nappy or an incontinence brief or incontinence underwear, or toilet-training garments.

In another aspect of the invention, there is provided a kit comprising:
(i) one or more moisture management pant undergarments for wicking bodily fluid away from a wearer's skin into a fluid trapping core component associated with the undergarment, the garment comprising
a pant body having a waistband, a front panel and a back panel connected by a pair of side wings, and a pair of leg openings having a crotch panel disposed therebetween, wherein inner surfaces of at least the crotch panel are lined with a brushed or otherwise textured surface, and
(ii) one or more removeable moisture management booster pads for positioning over the lined crotch panel, the booster pad comprising a fluid trapping core component and an external covering surrounding the core component, the covering comprising a moisture wicking fabric having a brushed or otherwise textured surface,
wherein contact between a lined surface of the crotch panel and a surface of the moisture wicking fabric of the booster pad, results in a friction or interference fit between the booster pad and the moisture wicking fabric lined crotch panel.

Desirably, in the kit, the one or more removeable moisture management booster pads are of different sizes and/or capacities.

Suitably, the moisture management pant undergarment is as defined in the first aspect.

Desirably, in the kit, the one or more removeable moisture management booster pads are defined in accordance with the second aspect of the invention.

Preferably, the moisture wicking fabric of the booster pad is the same as the moisture wicking fabric of the pant undergarment according to the first aspect.

Desirably, in the kit, the moisture wicking fabric of the booster pad and the pant undergarment comprises brushed polyester.

In a preferred kit, the moisture wicking fabric booster component is the same as the moisture wicking fabric of the pant undergarment of the invention. Desirably, the moisture wicking fabric of the booster pad is the same as the moisture wicking fabric of the pant undergarment of the first aspect. In a preferred kit, the moisture wicking fabric booster component and the moisture wicking fabric of the pant undergarment each comprise brushed polyester comprising a brushed or otherwise textured surface having fibers displaced from the fabric knit.

In a preferred kit, the moisture wicking fabric booster component and the moisture wicking fabric of the pant undergarment each comprise a brushed wool, particularly brushed Merino wool, a brushed wicking polyester, a brushed wicking polypropylene, a brushed wicking nylon, a brushed wicking micromodal, a brushed wicking bamboo, or composites thereof. Suitably, in a preferred kit, the moisture wicking fabric booster component and the moisture wicking fabric of the pant undergarment are each a brushed polyester composite, preferably containing more than 50% polyester and less than 50% spandex. Desirably, in a preferred kit, the moisture wicking fabric booster component and the moisture wicking fabric of the pant undergarment are each a brushed polyester composite comprising about 87% polyester and about 13% spandex.

In embodiments where the underwear garment is provided in the form of a nappy in a foldable configuration for folding into a nappy or an incontinence brief or incontinence underwear, or a toilet-training garments, it will be understood that the undergarment may include features such an elastic waist or an adjustable waist e.g., via adjustable press studs, buttons, hook & loop tape, clasps, hook & eyes or combinations thereof. This allows the undergarment to be folded and sized appropriately for the wearer's waist size. Likewise, the undergarment may include an adjustable crotch or gusset region, e.g., via adjustable press studs, buttons, hook & loop tape, clasps, hook & eyes or combinations thereof. This is particularly useful for nappies for children as the size can be adjusted appropriately as the child grows.

### Description of Preferred Embodiments

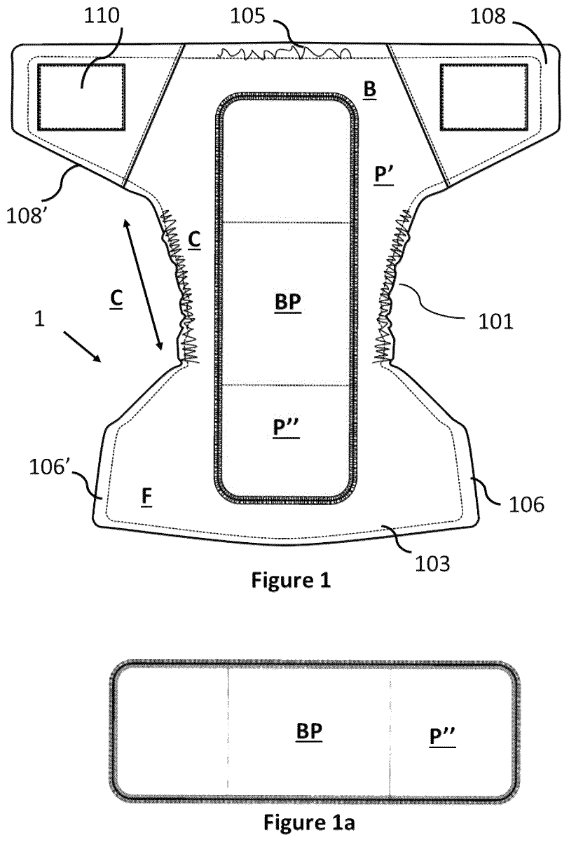

FIG. 1 shows a schematic of one example of the invention in the form of a fold up nappy or diaper (1) in a flat or unfolded configuration. The nappy (1) comprises a front panel (F) and back panel (B) as indicated generally by the arrows in the schematic. The general position of a crotch portion (C) is also indicated, the edges (101) of which are elasticated for comfort and assistance in avoiding leakages. Each of the front panel (F) and back panel (B) has a respective upper edge (103), (105) which in this embodiment is elasticated, again to improved comfort and fit during use.

Each of the front panel (F) and back panel (B) has pair of side wings, front left side wing (106) and front right side wing (106′) and back left side wing (108) and back right side wing (108′). Inner surface of each of the back left side wing (108) and back right side wing (108′) are provided with one component of a hook & loop tape fastener (110, 110′) which are attachable to an opposing component of the hook & loop tape fastener (now shown) positioned on the outer face of the front panel (F), preferably across the edge (103) of the nappy (1).

The inner (body facing) surfaces of the nappy (1) thus described are lined with, in this example, a polybrush fabric generally designated (P) in the schematic. If desired, a fluid trapping core could be included in the crotch panel region under the polybrush fabric liner. Likewise, the crotch panel could be adapted to include a floating liner comprising a fluid trapping core which itself could comprise a polybrush fabric liner to face the wearer's skin during use.

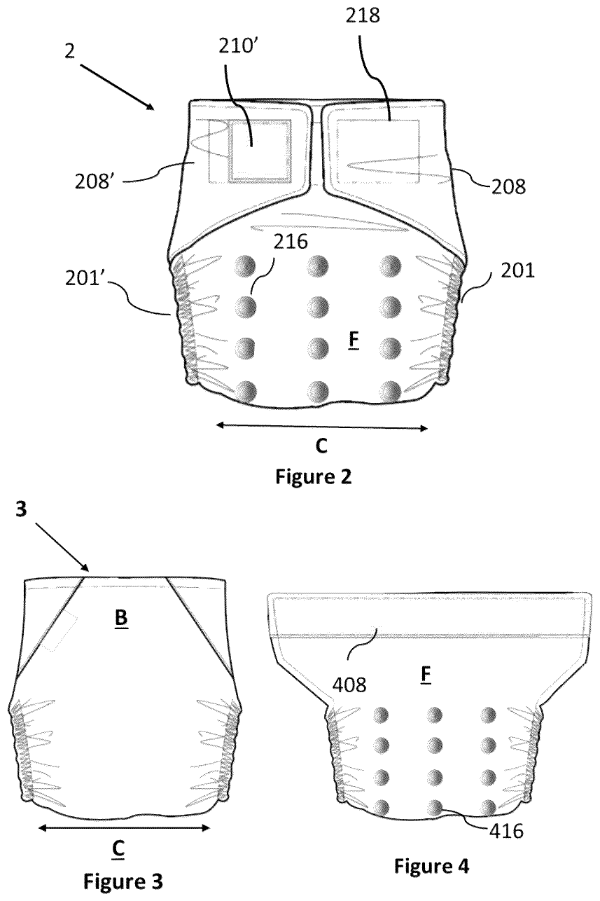

FIGS. 2 to 4 show various view of a pant undergarment of the invention in the form of a fold up nappy. However, the invention could equally well be embodiment as women's brief or underwear, men's brief or underwear, men's trunks or shorts, adult incontinence undergarments, children's toilet training pants, adaptive underwear or tween/teen incontinence underwear or the like.

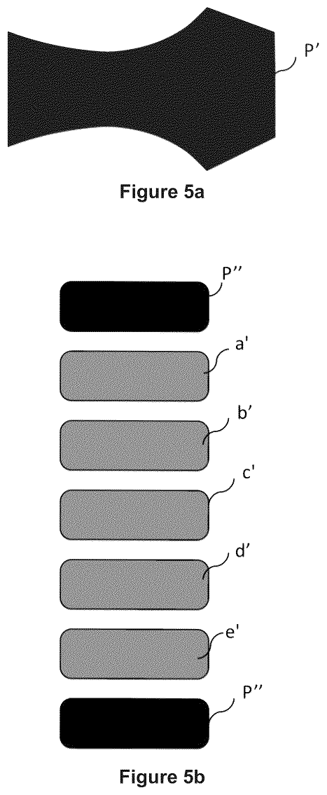

FIG. 5a shows the shape of an exemplary liner of the invention comprising a lining in the forms of a moisture wicking fabric having a brushed or otherwise textured surface. This moisture wicking fabric of the pant undergarment is designed to complement a moisture wicking fabric of a covering of a moisture management booster pad of the invention.

FIG. 5b shows an exploded view of the components of an example of a removeable moisture management booster pad of the invention booster pad of the invention for use with a nappy which shows the outer covering in the form of an upper covering layer and a lower covering layer which are a moisture wicking fabric having a brushed or otherwise textured surface. The inners layers, of which there are fiver layers in this example, comprise an absorbent polyester terry microfibre toweling fabric.

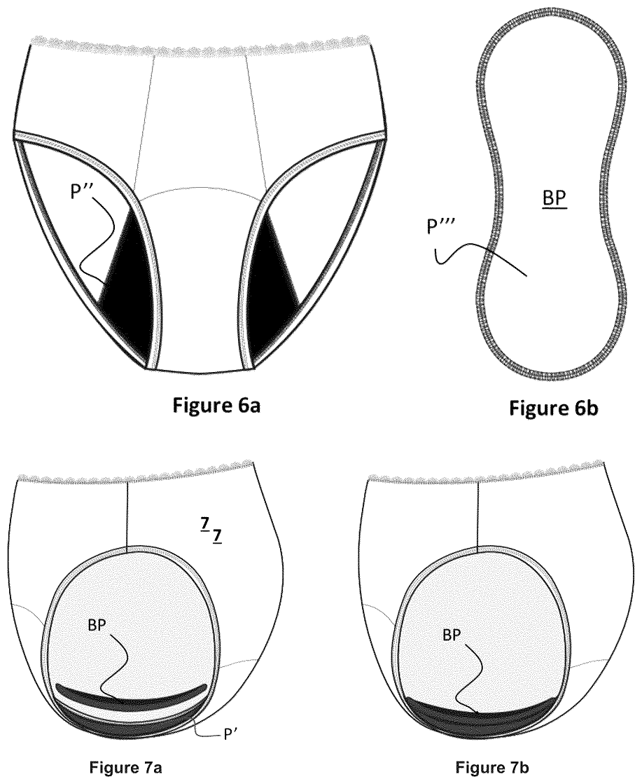

FIG. 6 illustrates a pair of women's underwear having the crotch portion provided with a lining in the forms of a moisture wicking fabric having a brushed or otherwise textured surface, as well as an example of a booster pad of the invention for use with a pair of women's underwear having a surface covering which in the form of an upper covering layer and a lower covering layer which are a moisture wicking fabric having a brushed or otherwise textured surface.

FIG. 7 shows the removeable moisture management booster pad of the invention being moved into position over the crotch portion of the underwear as well as a version where the booster pad is located in the crotch potion of the underwear in use.

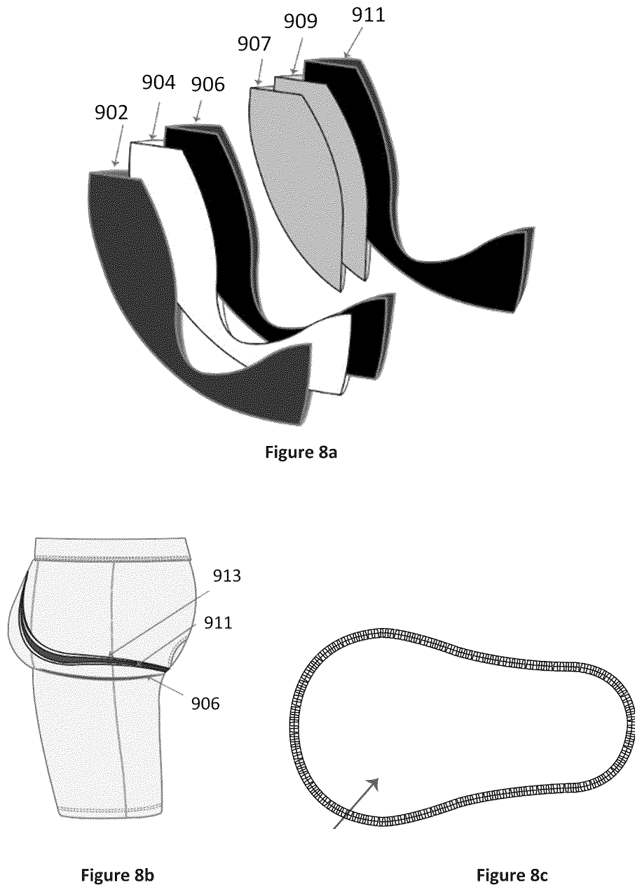

FIG. 8 shows (a) an exploded view of the fabric arrangement for a men's trunk style gusset layering, comprising a bamboo outer fabric (902), a waterproof layer (904) between the bamboo outer fabric (902) and a polybrush layer (906) forming the main construction of the trunk in the gusset/crotch area. FIG. 8a also shows the floating liner assembly comprising a first and second layer of microfibre terry (907, 909) and a polybrush inner layer (911) for contacting against the skin or a booster pad when included. FIG. 8b shows the full trunk outline with the main garment polybrush lining (906) shown, as well as the floating liner assembly provide with polybrush (911) facing the skin to be contacted when worn. FIG. 8c shows an exemplary booster pad for men's undergarments with additional volume capacity to on end of the pad.

### Nappy Drying Time Test

A nappy according to the invention with a floating liner component is sewn up inner leg to outer leg to create an attached gusset. A further nappy according to the invention was left with the desired floating liner/gusset in the floating configuration.

Each of the sample nappies were weighed while still dry before each nappy was fully saturated in water and both wrung out by hand three times to remove as much water as possible by hand. After this step, the wet nappies were weighed again before being hung out to dry. The nappy with the floating liner/gusset was hung in a way that ensures airflow between outer crotch portion/gusset and floating liner/gusset. Both are hung in same place, at the same point in time, under the same conditions.

After 12 and 24 hours drying time, the weight of the nappies is recorded. The results are provided below in Table 1 clearly demonstrating the nappy with the floating gusset has lost more water than the attached gusset nappy indicating the drying time benefit to the floating liner/gusset feature on drying.

| | Floating Gusset | Attached Gusset |
|---|---|---|
| Dry Pre-Testing | 148 g | 155 g |
| Wet, after wash and | 300 g | 310 g |
| wrung | | |
| After 12 hrs dry time | 239 g | 268 g |
| After 24 hrs dry time | 154 g | 181 g |

### Brush Fabric Cleaning Tests

A mixture of crunchy peanut butter, toothpaste and Keens curry powder, plus a little water was mixed and used in place of faeces. One teaspoon of this mixture was added to a series of boosters for testing wherein the booster had different surface fabrics as identified in the table below.

A 10 cm scrape of the mixture was prepared with a serrated knife. The solid materials were scraped off each booster and the booster surface was inspected and the visual appearance was recorded below.

Approximately 8 ml of water was added to each resultant stain on the booster. Each booster was rubbed for 20 second by hand and the booster surface was inspected and the visual appearance was recorded below.

Each booster was then washed under a tap while rubbing for 30 secs by hand, and the booster surface was inspected, and the visual appearance was recorded below.

Thereafter, each booster was machine washed for 15 minutes without detergent and the booster surface was inspected and the visual appearance was recorded below.

It is clear from the results of these tests that the polybrush fabric gave the best performance of all fabrics considered in terms of the faeces substitute tested. The faeces substitute can be scraped or wiped off easily, more so after gentle rubbing with all visible staining being removed after a 30 second hand wash and no staining or smells after a 15 minute machine wash.

| | Scrape | 20 Sec Rub | 30 Sec Hand Wash | 15 min machine wash |
|---|---|---|---|---|
| Polybrush | Good - | Good - | Good - particles have washed | Good - particles have washed off, |
| Booster | wipes off | Solids rub | off, no visible staining | no visible staining, smells clean |
| | easily | off | | |
| Grey | Bad - | Bad - | Bad - many embedded particles | OK, faint staining, smells of test |
| Fleece | engrained | engrained | remain, bad staining | solution, expect there are particles |
| Booster | | | | but less visible due to grey colour of |
| | | | | fabric |
| White | Bad - | Bad - | Bad - many embedded particles | Bad - some embedded particles |
| Fleece | engrained | engrained | remain, bad staining | remain, bad staining, smells of test |
| Booster | | | | solution |
| Brushed | Good - | Good - | OK - particles have washed off, | OK - particles have washed off but |
| Pile | wipes off | Solids rub | bad staining | bad staining, smells of test solution |
| Bamboo | easily | off | | |
| Booster | | | | |
| Jersey | Good - | Good - | OK - particles have washed off, | Bad - particles have washed off but |
| Knit | wipes off | Solids rub | bad staining | really bad staining, smells of test |
| Cotton | easily | off | | solution |
| Booster | | | | |

1. A launderable moisture management pant undergarment comprising:
(a) a pant body comprising leg openings having a crotch panel disposed therebetween, wherein a surface of at least the crotch panel are lined with a first moisture wicking fabric comprising a brushed or otherwise textured surface having fibers displaced from the fabric knit, and
(b) a removeable moisture management booster pad for positioning over the lined crotch panel, the booster pad comprising:
an external covering surrounding a fluid trapping core component of the booster pad, the external covering comprising a second moisture wicking fabric comprising a brushed or otherwise textured surface having fibers displaced from the fabric knit, and
wherein the fluid trapping core component of the booster pad traps bodily fluid wicked away from the wearer's skin by the second moisture wicking fabric of the booster pad;
wherein
physical contact between a first surface of the moisture wicking fabric lined crotch panel of the pant undergarment and a second surface of the moisture wicking fabric of the external covering of the booster pad, results in a friction, non-slip or interference fit between the booster pad and the moisture wicking fabric lined crotch panel.

2. The moisture management pant undergarment according to claim 1, wherein the removeable moisture management booster pad is free of fastener devices for releasably attaching the booster pad to the pant body.

3. The pant undergarment of claim 1, wherein the first moisture wicking fabric lining the crotch portion and the second moisture wicking fabric of the covering of the booster pad are the same fabric or are different fabrics.

4. (canceled)

5. The pant undergarment of claim 1, wherein one or more of the first and second moisture wicking fabrics comprises a brushed wool, a brushed wicking polyester, a brushed wicking polypropylene, a brushed wicking nylon, a brushed wicking micromodal, a brushed wicking bamboo, a brushed cellulosic fabric, a brushed cotton, or composites or blends thereof.

6. The pant undergarment of claim 1, wherein the moisture wicking fabric is a brushed fabric comprising a brushed polyester or a brushed polyester composite, preferably containing more than 50% polyester and less than 50% spandex.

7. The pant undergarment of claim 1, wherein an optional second fluid trapping core component is attached to the floating liner or to an upper side of the crotch portion or wherein an optional second fluid trapping core component is attached to the underside of the floating liner or is removably attached to the floating liner.

8. (canceled)

9. The pant undergarment of claim 7, wherein at least a portion of the crotch panel is overlaid with a floating liner fixedly attached to the pant body and adapted to accommodate the optional second fluid trapping core component which traps bodily fluid wicked away from the wearer's skin, wherein at least one or more portions of the floating liner and the crotch panel can be physically separated from each other to expose the optional second fluid trapping core component to aid drying.

10. The pant undergarment of claim 9, wherein at least one or more portions of the floating liner are unattached to the pant body allowing physical separation of the optional second fluid trapping core component from the crotch portion.

11. The pant undergarment of claim 1, wherein edges of the pant body at the leg openings are elasticated.

12. The pant undergarment of claim 1, wherein at least a portion of the floating liner comprises the first moisture wicking fabric comprising a brushed or otherwise textured surface.

13. The pant undergarment of claim 1, wherein the fluid trapping core component comprises one or more layers of one or more layers of an absorbent, quick drying fabric.

14. The pant undergarment of claim 1, wherein one or more of the first and second fluid trapping core components comprise an absorbent, quick drying fabric manufactured from a cellulosic material.

15. The pant undergarment of claim 1, wherein at least a portion of the crotch panel includes a moisture barrier component to prevent moisture leaking outside the pant during use.

16. (canceled)

17. The pant undergarment of claim 15, wherein the moisture barrier component is a fabric which comprises a waterproof or water-resistant laminate film or a chemical coating, rendering the fabric moisture proof, moisture resistant fabric or moisture repellent.

18. The pant undergarment of claim 1, in the form of a launderable nappy, a launderable incontinence brief, launderable incontinence underwear, a launderable incontinence trunk, launderable incontinence pantyhose, launderable incontinence leggings, launderable incontinence sports shorts, launderable toilet-training garments, launderable adaptive underwear, launderable tween/teen incontinence underwear, or a launderable period management underwear.

19. The pant undergarment of claim 1, in a pull up confirmation or in a foldable configuration to be folded into a nappy or an incontinence brief or incontinence underwear, or a toilet-training garment.

20. (canceled)

21. (canceled)

22. (canceled)

23. The pant undergarment of claim 1, supplied as part of a kit comprising:
(i) one or more removeable moisture management booster pads, the booster pad comprising:
an external covering surrounding a fluid trapping core component, the external covering comprising a second moisture wicking fabric comprising a brushed or otherwise textured surface comprising a brushed or otherwise textured surface having fibers displaced from the fabric knit,
wherein the fluid trapping core component of the booster pad traps bodily fluid wicked away from the wearer's skin by the moisture wicking fabric into the core of the booster pad,
wherein
physical contact between a first surface of the moisture wicking fabric lined crotch panel of the pant undergarment and a second surface of the moisture wicking fabric of the external covering of the booster pad, results in a friction, non-slip or interference fit between the booster pad and the moisture wicking fabric lined crotch panel; and
(ii) one or more moisture management pant undergarments each comprising:
a pant body comprising leg openings having a crotch panel disposed therebetween, wherein inner surfaces of at least the crotch panel are lined with a further moisture wicking fabric comprising a brushed or otherwise textured surface having fibers displaced from the fabric knit.

24. (canceled)

25. (canceled)

26. The pant undergarment of claim 5, wherein one or more of the first and second moisture wicking fabrics comprises brushed Merino wool.

27. The pant undergarment of claim 13, wherein the absorbent, quick drying fabric is a polyester microfibre fabric.

28. The pant undergarment of claim 13, wherein the absorbent, quick drying fabric is a microfibre toweling comprising about 80% polyester and about 20% polyamide.
